(19) United States
(12) Patent Application Publication
(10) Pub. No.: US 2012/0118282 A1
(43) Pub. Date: May 17, 2012

(54) INSULATING AND HEATING SAFETY DEVICE APPLICABLE TO WATERPROOF CLOTHES AND WORK CLOTHES

(75) Inventor: José Maria Santiago Fontaiña, La Coruna (ES)

(73) Assignee: SAVE-DUMMY, S.L., Riviera A Coruna (ES)

(21) Appl. No.: 13/321,140

(22) PCT Filed: May 7, 2010
(86) PCT No.: PCT/ES2010/000202

§ 371 Date: Feb. 3, 2012

(30) Foreign Application Priority Data
May 18, 2009 (ES) P200901238

(51) Int. Cl. A61F 7/08

(52) U.S. Cl. 126/204

(57) ABSTRACT

A safety device can easily be incorporated into any type of waterproof clothes and work clothes and is designed for protecting people who accidentally fall into the sea, insulation against low water temperatures, as well as a heat source that considerably prolongs survival time in low-temperature water being provided. The device includes a main ring-shaped bag (1), the outermost part of which is provided with long, narrow secondary bags (5); and a check valve (3) arranged on the surface of the main bag which contains sodium which, upon contact with water, causes a chemical reaction that seals possible points at which water could enter the clothing and further provides a heat source for the body.

### OBJECT OF THE INVENTION

The present invention relates, as shown by the title of the present specification, to a safety device which can easily be incorporated to any type of waterproof clothes, work clothes and in general, to any type of clothing, which is specifically designed for protecting people who accidentally fall into the sea, insulation against low water temperatures, as well as a heat source that considerably prolongs the survival time in low-temperature water being provided while waiting for rescue.

More specifically, the object of the invention consists of a ring-shaped bag, which has different long, narrow bags connected to it at several points on its surface. The ring-shaped bag is conceived to be integrated in the extremities of the garment, whereas the long bags must be attached to the clothes lining such that they cover, the entire inner surface of the clothing, in the most uniform manner as possible.

Upon activating the device, once it has been introduced into the water, the ring-shaped bag will inflate, such that it creates a seal with the user body thus preventing the entrance of water and providing a certain insulation. Simultaneously, a chemical reaction will be started with heat generation inside the long bags such that a hot tubing network which will provide heat to the user for a time is created. The combined effect of both bags allows heating the water which has been trapped inside the clothing such that the user is completely protected from the danger of hypothermia due to prolonged exposure to low temperatures.

### FIELD OF APPLICATION

The field of application of this invention is within the safety equipment manufacturing industry, especially within the field of maritime safety.

### BACKGROUND OF THE INVENTION

The problems associated to the use of traditional impermeable life jackets are widely known by any person with certain experience in the marine sector: given the discomfort involved in wearing them while working, it is very common to find that the operators work without wearing them, despite the existing obligation to that respect. In addition, the life jackets only assure the buoyancy of the person wearing it, not providing any protection against low temperatures, this leading to the fact that many shipwrecked victims die due to hypothermia in a few hours, even minutes, after falling into the water while he/she is being searched for.

In many occasions, the rescue of a person who falls into the sea is delayed as a consequence of bad climatological conditions, or even when said fall is not witnessed by the rest of the crew, such that the rescue is not started until the absence of that person is detected, a very valuable time which may determine the difference of finding him/her alive or dead is already lost.

Due to the foregoing, it is desirable to produce a device which will allow prolonging the survival time of a person who falls into sea against the existing low temperatures, and which simultaneously provide features such as those which will allow integrating it in the common work clothing, such that the protection is provided by the device if one accidentally falls into the sea, even when the user is unconscious, or in situations where the lack of time does not allow adopting protection measures.

Patent with reference number P9900023 (Universidad Politécnica de Catalunya) describing an impermeable and thermally insulated life jacket is disclosed by the holder of the present specification. As any person skilled in the art could easily verify, the jacket described in said document although intended to assure the buoyancy and the insulation of the person wearing it, it is necessary to provide a certain time to get into the jacket, such that it is not valid for situations described in the paragraph above, the main function thereof being different from that of the device described herein.

The existence of the French patent FR2258137 (Philippe Verger) describing a ring-shaped bag device which, inflated through a valve arranged on its outside, allows sealing the possible points at which water could enter the clothing by pressing against the body of the user, is also known. Said bags are integrated in the clothes of the user similarly to that described in the present specification, although the inflating system is completely different, significant difference with respect to the invention described herein being further present, where this device does not provide any heat source, besides lacking of a means for distributing said heat.

Another problem raised in the aforementioned situation where a person accidentally falls into the water, and where it is not solved with any currently known device or type of clothing, is derived from the fact that the first action performed after falling into the water tends to be removing the boots to facilitate floating. This leaves the skins without any protection, being an important path of body heat loss, therefore finally the resistance time of the person is significantly reduced. A variant of the device described (also applicable to the hands) solves this situation by means of introducing the pertinent variants described below.

The present insulating safety device with heat source applicable to waterproof clothes and work clothes is thus designed as an obvious novelty, further solving the problems which have been previously mentioned and offering safety performances better than those of the systems existing in the current state of the art.

### SUMMARY OF THE INVENTION

The device is formed by a main ring-shaped bag made of plastic material such as PVC or the like the outer most part of which has (the part opposite the part which is intended to contact the user body once the bag is inflated) a plurality of long, narrow bags. The ring-shaped bag is designed to be integrated in the clothes, such that once inflated it presses against the body of the user, preventing the entrance of water therein.

The inside of the main bag will contain a specific amount of sodium in solid state, being able to be introduced in the forms of sheets, granules or any other valid form. Likewise, inside each of the long bags it will contain a specific amount of distilled water, which could not go to the main bag due to fact that a cover will be provided in the attachment between both bags which will separate them. This cover will be made such that an increase of pressure inside the bag will break it, any valid material being used for this function.

The main bag will also have on its outer surface a small check valve, which will allow the passage of a small amount of water to the inside but prevent its exit through the same opening by preventing the flow in that direction. This valve will also be closed when the pressure inside the bag exceeds the pressure existing outside.

The operation process of the device starts when the latter is introduced into the water. When a depth of a few centimeters is reached, the check valve incorporated to the main bag will open allowing the passage of the water, which will contact the solid sodium existing therein, such that the following widely known chemical reaction is produced:

Na+H₂O→Na OH+H₂

The hydrogen produced in the reaction will be released into the bag, such that the latter will start to inflate. At the same time, the sodium hydroxide (commonly known as caustic soda), as it is being produced will start to mix with the water present, process in which a high amount of heat is generated.

What is searched for by causing the mentioned chemical reaction is the generation of a stable substance either in liquid or solid state from the mixture with water such that a gas together with a relatively stable heat emission is obtained. The elements belonging to the alkaline metal group cause this reaction, the sodium being considered as the element offering the best performances. However, any other element or chemical compound capable of providing a gas together with heat emission upon mixing with water would be suitable for the embodiment of the present invention.

The pressure increase in the main bag will serve to prevent the passage of more water to the inside by closing the check valve, and it will also break the covers connecting the long bags, such that again due to the effect of the pressure increase, the heat generating water and soda mixture will be distributed throughout most part of the bags, heating the user of the device. The water contained in the long bags will allow the continuity of the reaction until all the soda generated has been completely exhausted.

Since the present device will be made in different sizes to enable adapting to different positions inside the user clothing (cuffs, belt, trousers leg hems . . . ), in each of the models the necessary amounts of sodium and water to be introduced into the main bag will be previously calculated, such that suitable floating pressures are obtained and the existing risk of explosion in the event that the heat generated by the soda may be able to react with the hydrogen present is also prevented. In all cases, the small amounts necessary to make the device allow manufacturing a bag which has no problem resisting a possible accidental explosion, which could not have any consequence for the user or for the operation of the device.

A variant of the device described, conveniently adapted to the ankles or the wrists, will incorporate small duly folded bags attached to it to occupy the least volume possible, allowing to be used as socks or mittens where appropriate, such that the user can put them on once they are extended. One or more of the long bags will be attached to these bags such that the heat flow will also reach the part of the body which it covers. These new bags will be manufactured in an impermeable and thermally insulating material which minimizes heat loss produced through hands and feet.

### DESCRIPTION OF DRAWINGS

To complement the description which is being made and in order to facilitate a better understanding of the features of the invention, several drawings are attached as an integral part of the present specification in which the following are described in an illustrative and non-limiting manner:

FIG. 1 shows a simplified view of the device.

FIG. 2 shows a perspective view of the inflated device.

FIG. 3 shows the device in condition of use.

FIG. 4 shows the device modified for hands and feet .

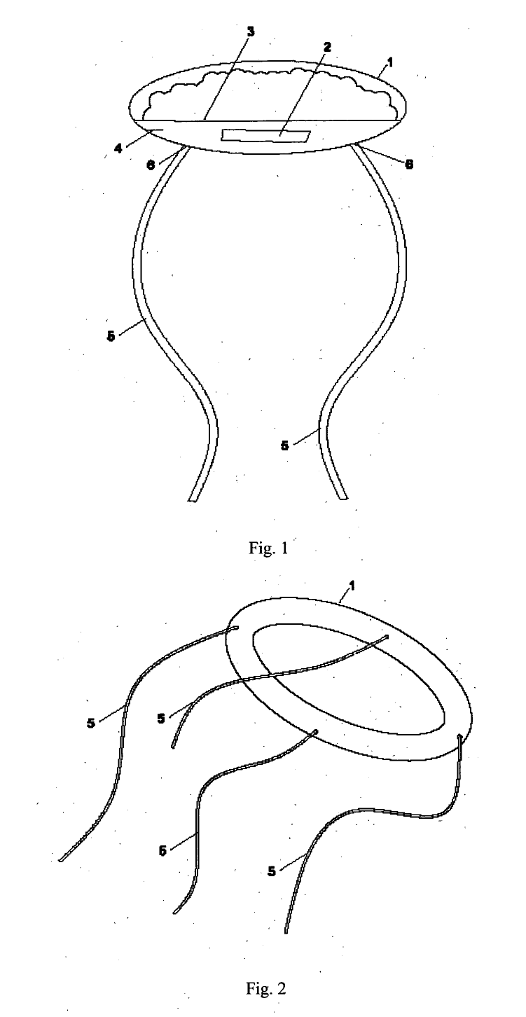

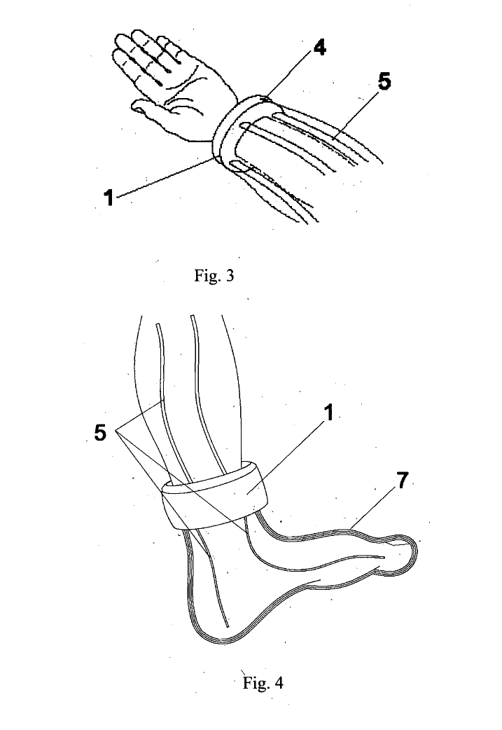

### PREFERRED EMBODIMENT OF THE INVENTION

In view of these drawings, the present invention is illustrated with the following embodiment, not attempting at all to limit the scope thereof.

In a preferred embodiment, the device is formed by a main ring-shaped bag (1) and made of PVC having on its outer most part long, narrow secondary bags (5).

The main bag (1) has on its surface a check valve (4) formed by a simple flange with flap that gives way with respect to the outer water pressure but prevents the exit of any fluid from the bag. This flange will be made such that a small pressure is necessary to allow the passage of the water (which will be translated into the fact that the water does not penetrate until several centimeters of depth are reached).

The connection of the main bag (1) with the secondary (5) is also blocked by a cover made by means of a thin PVC layer (6) which will be broken with a pressure increase inside the device. The secondary bags (5) have a small amount of water therein, to facilitate the continuity of the chemical heat generating reaction.

A small amount of solid sodium granule which will contact with the water when the latter penetrates will be arranged inside the main bag.

The device, once integrated in the clothing, will be hidden from view since it is introduced in the inner lining, suitably distributing the secondary bags (5) and fixing them to the clothes by means of any valid method such as adhesive or the like.

FIG. 4 shows an alternative embodiment of the device adapted for hands and feet. The auxiliary bags (7) having two secondary bags (5) to provide heat are observed in this drawing. These bags are attached by means of adhesive, any other system which is effective being valid, or this connection even not being needed if the sufficient number of secondary bags (5) is attached to it as to provide sufficient fastening.

Having sufficiently described the nature of the present invention as well as the way of putting it into practice, it is not considered necessary to better explain it for any person skilled in the art to understand its scope and the advantages resulting from it, it remains to be stated that within its essentiality, it could be put into practice in other embodiments differing in detail from that indicated by way of example, such as the use thereof in other type of garments or work clothes, and the protection claimed will be equally achieved provided that its essential principle is not altered, changed or modified.

1. An insulating and heating safety device applicable to waterproof clothes and work clothes comprises a main ring-shaped bag the outer most part of which has of a plurality of long, narrow secondary bags, and a check valve arranged on the surface of the main bag.

2. The insulating and heating safety device applicable to waterproof clothes and work clothes according to claim 1, wherein the main bag contains a specific amount of sodium in solid state.

3. The insulating and heating safety device applicable to waterproof clothes and work clothes according to claim 1, further comprising covers with a bursting resistance lower than the rest of the components of the device arranged in the a connection of the main bag to the secondary bags.

4. The insulating and heating safety device applicable to waterproof clothes and work clothes according to claim 1, wherein the secondary bags are uniformly distributed inside the clothing wherein the safety device is integrated.

5. The insulating and heating safety device applicable to waterproof clothes and work clothes according to claim 1, wherein the secondary bags have distilled water therein.

6. The insulating and heating safety device applicable to waterproof clothes and work clothes according to claim 1, further comprising an auxiliary bag is attached to the main bag.

7. The insulating and heating safety device applicable to waterproof clothes and work clothes according to claim 6, wherein has one or more secondary bags are attached to the auxiliary bag.
